(19) United States
(12) Patent Application Publication
(10) Pub. No.: US 2016/0038349 A1
(43) Pub. Date: Feb. 11, 2016

(54) Absorbent Feminine Hygiene Device

(71) Applicant: Amalia Mainali, Escondido, CA (US)

(72) Inventor: Amalia Mainali, Escondido, CA (US)

(21) Appl. No.: 14/801,920

(22) Filed: Jul. 17, 2015

Related U.S. Application Data
(60) Provisional application No. 62/035,598, filed on Aug. 11, 2014.

(51) Int. Cl. A61F 13/472; A61F 13/56; A61F 13/84

(52) U.S. Cl. CPC A61F 13/472; A61F 13/8405; A61F 13/5611; A61F 2013/4708

(57) ABSTRACT

An absorbent feminine hygiene device for absorbing fluid and odor from vaginal, menstrual, or incontinence discharge. The absorbent feminine hygiene device includes an elongated pad having a pad member connected to a curved member, wherein both members include a first end opposing a second end, an adhesive rear surface, and an absorbent front surface. The pad member is rectangular in shape and can be positioned over a user's vagina, wherein the first end thereof is connected to the second end of the curved member. The curved member extends upwards from the pad member in order to be positioned between a user's buttocks. The front surface of the curved member includes a ridge thereon, which can be placed between the buttocks of the user so as to absorb discharge that can leak towards the user's buttocks when she is lying down or sitting.

### CROSS REFERENCE TO RELATED APPLICATION

This application claims the benefit of U.S. Provisional Application No. 62/035,598 filed on Aug. 11, 2014. The above identified patent application is herein incorporated by reference in its entirety to provide continuity of disclosure.

### BACKGROUND OF THE INVENTION

### Field of the Invention

The present invention relates to absorbent feminine hygiene devices. More specifically, the present invention provides an absorbent feminine hygiene device comprising a pad member and a curved member, wherein the device is adapted to be positioned over a user's vagina and along the user's buttocks in order to absorb fluid and odor therefrom.

Many women experience a heavy flow during their menstrual cycle which may cause stains to develop on one's clothes, sheets, furniture and the like if not wearing the proper protection. These occurrences are extremely embarrassing and uncomfortable, especially when at work or in public places. Conventional sanitary pads and tampons do not offer adequate and full protection, particularly when lying down or in a seated position. The use of these conventional devices becomes inefficient after a prolonged period of time due to leakage that occurs towards the user's buttocks region. Discharge, blood, or other fluid can travel directly towards the buttocks area, thereby bypassing the conventional pad and leak onto an individual's undergarments. Therefore, there exists a need in the prior art for an absorbent feminine hygiene device that extends upward towards the buttocks region, thereby protecting the user when experiencing heavy flow or when in a seated or lying position.

It is submitted that the present invention substantially diverges in design elements from the prior art and consequently it is clear that there is a need in the art for an improvement to existing absorbent feminine hygiene devices. In this regard the instant invention substantially fulfills these needs.

### SUMMARY OF THE INVENTION

In view of the foregoing disadvantages inherent in the known types of absorbent feminine hygiene devices now present in the prior art, the present invention provides a new absorbent feminine hygiene device wherein the same can be utilized for providing convenience for the user when absorbing fluid and odor from vaginal, menstrual, or incontinence discharge.

It is therefore an object of the present invention to provide a new and improved absorbent feminine hygiene device that has all of the advantages of the prior art and none of the disadvantages.

It is another object of the present invention to provide an absorbent feminine hygiene device comprising an elongated pad adapted to extend from a user's pubic region, covering the vagina, and terminating at the upper end of the user's buttocks in order to absorb fluid and odor therefrom.

Another object of the present invention is to provide an absorbent feminine hygiene device wherein the elongated pad comprises a pad member having a first end opposing a second end, wherein the pad member is rectangular in shape and adapted to extend from a user's pubic region and terminate at the user's buttocks.

Yet another object of the present invention is to provide an absorbent feminine hygiene device wherein the elongated pad further comprises a curved member connected to the second end of the pad member, wherein the curved member includes a ridged front surface adapted to be placed between a user's buttocks in order to provide leak protection.

Yet another object of the present invention is to provide an absorbent feminine hygiene device wherein the rear surface of the elongated pad comprises one or more adhesive strips so as to allow the elongated pad to adhere to a user's undergarment.

Another object of the present invention is to provide an absorbent feminine hygiene device that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the absorbent feminine hygiene device.

FIG. 2 shows a side view of an embodiment of the absorbent feminine hygiene device.

FIG. 3 shows a rear view of an embodiment of the absorbent feminine hygiene device.

### DETAILED DESCRIPTION OF THE INVENTION

Reference is made herein to the attached drawings. Like reference numerals are used throughout the drawings to depict like or similar elements of the absorbent feminine hygiene device. For the purposes of presenting a brief and clear description of the present invention, the preferred embodiment will be discussed as used for absorbing fluid and odor from vaginal, menstrual, or incontinence discharge. The figures are intended for representative purposes only and should not be considered to be limiting in any respect.

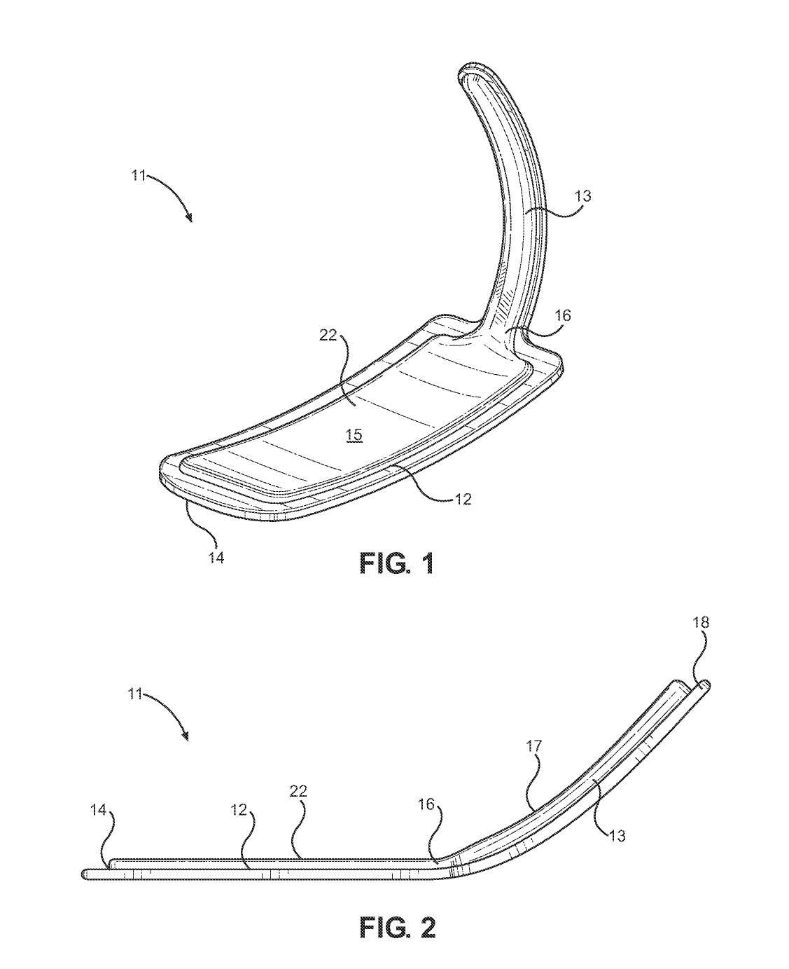

Referring now to FIG. 1 there is shown a perspective view of an embodiment of the absorbent feminine hygiene device. The absorbent feminine hygiene device 11 comprises an elongated pad 12 adapted to extend from a user's pubic region to the upper end of the user's buttocks. The elongated pad 12 is adapted to provide a sanitary napkin, pad, panty liner, or the like that can absorb discharge, blood, or other fluids while preventing leakage therefrom. The elongated pad 12 comprises a front surface 15 and a rear surface, wherein the front surface 15 is adapted to be positioned against the user's body. The rear surface comprises an adhesive layer and a film that covers the adhesive layer, wherein the film is removable prior to use. The adhesive layer of the rear surface is adapted to be adhered to the inside of a user's undergarment in order to secure the absorbent feminine hygiene device in place. The front surface 15 of the elongated pad 12 can be composed of one or more layers of any suitable absorbent material, such as cotton or rayon. The rear surface of the elongated pad 12 is preferably composed of a liquid impermeable material to prevent leakage and seepage therethrough.

The elongated pad 12 further comprises a pad member 22 and a curved member 13, wherein the pad member 22 is adapted to position around a user's vagina so as to absorb fluid and odor therefrom. The pad member 22 is flexible due to the flexibility of the material thereof and can be folded in a compact configuration for storing in a purse or pocket. The pad member 22 is rectangular in shape and comprises a first end 14 opposing a second end 16. The pad member 22 comprises a concave curvature and the second end 16 is connected to the curved member 13. The pad member 22 and the curved member 13 are integral to one another. In some embodiments, the lateral side of the pad member 22 are curved inward in order to correspond to the shape of the crotch area of a user's undergarment.

Now referring to FIG. 2, there is shown a side view of an embodiment of the absorbent feminine hygiene device. The curved member 13 is elongated and comprises a first end 18 opposing a second end, wherein the second end is connected to the second end of the pad member 22. The upper surface of the curved member 13 protrudes outwards in order to provide absorbent material to comfortably fit between the buttocks of a user. The protruded upper surface is adapted to expand once fluid is absorbed therein.

In the illustrated embodiment, the protrusion is a ridge 17 disposed on the front surface of the curved member 13. The ridge 17 comprises an elevated absorbent front surface that narrows towards the top thereof. The elevated surface of the ridge 17 provides additional material along the curved member 13 so as to allow the curved member 13 to absorb more fluid therein. Furthermore, the narrowing of the ridge 17 towards the top thereof is adapted to allow the ridge 17 to be comfortably positioned between a user's buttocks so as to avoid having a wider material positioned therebetween. The ridge 17 extends the length of the curved member 13 and is adapted to absorb fluid and odor that may travel from the pad member 22 or come directly from the user.

When a user is in a seated or lying position, the fluid travels towards the user's buttocks. The ridge 17 is adapted to allow the curved member 13 to fit securely between the user's buttocks and provide additional thickness to absorb fluid. The ridge 17 is further adapted to rest flush against the user's skin. In other embodiments, the upper surface of the curved member 13 comprises any suitable cross section, such as a semi-circular or circular, as long as the upper surface is adapted to comfortably fit within the user's buttocks and absorb liquid therein.

The curved member 13 is disposed centrally on the second end 16 of the pad member 22 and extends upwards therefrom. The curved member 13 comprises a curve so as to correspond to the curvature of a user's buttocks. The curved shape prevents the curved member 13 from collapsing and becoming removed from the user's buttocks while in use. The curve is able to maintain its shape due to the rigidity of the additional absorbent material used to compose the front surface thereof, which includes the ridge 17. The second end of the curved member 13 is adapted to terminate at the top of a user's buttocks.

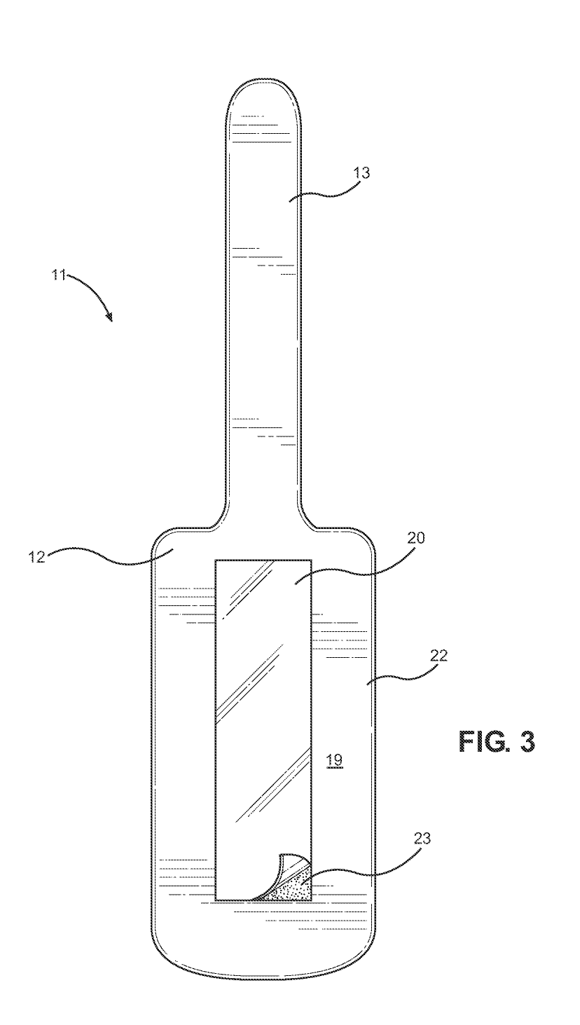

Referring now to FIG. 3, there is shown a rear view of an embodiment of the absorbent feminine hygiene device. The rear surface 19 of the elongated pad 12, including the pad member 22 and curved member 13, is embedded with a scent, such as a fruit scent or a flower scent, in order to mask unwanted odors. In other embodiments, the absorbent feminine hygiene device 11 is odorless. The rear surface 19 of the pad member 22 further comprises one or more elongated adhesive strips 23 adapted to adhere and removably secure the pad member 22 to the interior of a user's undergarment. Any suitable adhesive can be used, such as pressure sensitive adhesive. There is an adhesive strip 23 on the rear surface 19 of the pad member 22, wherein the adhesive strip 23 is rectangular in shape. In some embodiments, the entire rear surface 19 is covered by an adhesive layer. In other embodiments, the adhesive strip 23 can be any suitable shape as long as the rear surface is able to adhere to the interior of a user's undergarment.

The adhesive strips 23 are covered by a film 20 in order to protect the adhesive from adhering to another surface prior to use. The dimension of the film 20 is the same as the dimension of the adhesive strip 23 in order to protect the entire surface thereof. In operation, the film is removed from the adhesive strip 23 and subsequently disposed of. In some embodiments, the pad member 22 comprises a pair of flaps extending from the lateral sides of the pad member, wherein the rear surface of the flaps include an adhesive thereon and the front surface does not include an absorbent material. The flaps are adapted to fold under the pad member 22 and adhere to the exterior of the crotch area of a user's undergarment. In this way, the elongated pad 12 is further secured in position while in use.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An absorbent feminine hygiene device, comprising:
an elongated pad comprising a front surface and a rear surface, wherein said rear surface is adapted to removably secure to a user's undergarment;
wherein said elongated pad comprises a pad member connected to a curved member;
wherein said pad member comprises a first end opposing a second end, wherein said pad member is adapted to extend from a user's pubic region and terminate at a user's buttocks;
wherein said curved member comprises a first end opposing a second end, wherein said first end is connected to said second end of said pad member;
wherein said curved member comprises a protrusion thereon adapted to be positioned between said user's buttocks.

2) The absorbent feminine hygiene device of claim 1, wherein said curved member extends upward from said pad member.

3) The absorbent feminine hygiene device of claim 1, wherein said pad member is rectangular in shape.

4) The absorbent feminine hygiene device of claim 1, further comprising one or more elongated adhesive strips disposed on said rear surface of said elongated pad.

5) The absorbent feminine hygiene device of claim 4, wherein each of said one or more elongated adhesive strips is covered by a removable film.

6) The absorbent feminine hygiene device of claim 1, further comprising a scent embedded on said rear surface of said elongated pad adapted to mask odor.

7) The absorbent feminine hygiene device of claim 1, wherein said front surface of said elongated pad is composed of an absorbent material.

8) The absorbent feminine hygiene device of claim 1, wherein said protrusion is a ridge.
